(19) United States
(12) Patent Application Publication
(10) Pub. No.: US 2016/0200699 A1
(43) Pub. Date: Jul. 14, 2016

(54) GLYCIDOL PREPARATION

(71) Applicants: Fergal COLEMAN, Belfast Antrim (GB); John HOLBREY, Belfast Antrim (GB); Kenneth SEDDON, Belfast Antrim (GB); Martin ATKINS, Belfast Antrim (GB); Yoan DELAVOUX, Belfast Antrim (GB); Jeni TOLAND, Belfast Antrim (GB); Ting YAN, Belfast Antrim (GB)

(72) Inventors: Fergal Coleman, Belfast (GB); John Holbrey, Belfast (GB); Kenneth Seddon, Belfast (GB); Martin Atkins, Belfast (GB); Yoan Delavoux, Belfast (GB); Jeni Toland, Belfast (GB); Ting Yan, Belfast (GB)

(21) Appl. No.: 14/910,835

(22) PCT Filed: Aug. 8, 2014
(86) PCT No.: PCT/GB2014/052435

§ 371 Date: Feb. 8, 2016

(30) Foreign Application Priority Data
Aug. 8, 2013 (GB) 1314254.2
Aug. 8, 2013 (GB) 1314257.5
Aug. 8, 2013 (GB) 1314258.3

(51) Int. Cl. C07D 301/02

(52) U.S. Cl. CPC C07D 301/02

(57) ABSTRACT

This invention relates to an improved one-pot synthetic process for the preparation of glycidol from the reaction of glycerol and dimethyl carbonate. More specifically, the invention relates to a one-pot synthetic process for the preparation of glycidol comprising the reaction of glycerol and dimethyl carbonate in the presence of an ionic liquid catalyst having the formula: [Cat+][X] wherein: [Cat] represents one or more cationic species, and [X] represents one or more anionic species; wherein the reaction is conducted at a temperature of from 100° C. to 160° C. and wherein the molar ratio of glycerol to dimethylcarbonate is from 1:4 to 1:10.

This invention relates to an improved one-pot synthetic process for the preparation of glycidol from the reaction of glycerol and dimethyl carbonate. More specifically, the invention relates to a process where the synthesis of glycidol is conducted in the presence of specifically selected ionic liquids using specifically selected reaction conditions.

Glycidol (GD) is a known compound which has a number of valuable industrial uses. It is known to have properties making it useful in stabilizers, plastics modifiers, surfactants, gelation agents and sterilizing agents. Furthermore, GD is known to be useful as an intermediate in the synthesis of glycidyl ethers, esters, amines, as well as glycidyl carbamate resins and polyurethanes. It has therefore found application in a variety of industrial fields including textile, plastic, pharmaceutical, cosmetic and photochemical industries.

Known commercial processes for the preparation of GD include epoxidation of allyl alcohol using hydrogen peroxide and a tungsten-oxide based catalyst, and the reaction of epichlorohydrin with bases. However, there are drawbacks relating to these processes. For instance, the epoxidation of allyl alcohol involves several process steps and suffers problems relating to decomposition of the catalyst. Meanwhile, the high cost of raw materials and/or the management of waste by-products are a concern in both cases.

Glycerol (GL) is produced in large quantities as a by-product in the production of biodiesels. With an increasing focus on the use of biofuels to at least partly replace petroleum fuels, the production of glycerol has increased to levels far higher than current demand. As a result, GL is a cheap and readily available material, particularly in countries where production of biofuels is prevalent, and there has been an increased focus on the development of suitable applications of GL.

S. M. Gade et al., Catalysis Communications, 27, 2012, pages 184 to 188 (hereinafter 30 referred to as “Gade et al”), reports an alternative one-pot synthesis of GD from GL and dimethyl carbonate (DMC) under mild conditions using an ionic liquid catalyst.

The process reported by Gade et al involves transesterfication of DMC with GL to form glycerol carbonate (GC) as an intermediate before decarboxylation thereof affords GD, as well as carbon dioxide as a by-product, as illustrated in the reaction scheme below:

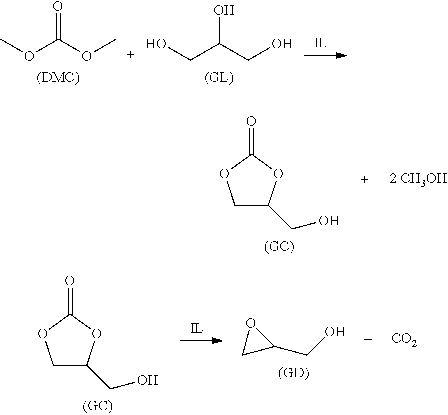

It is reported initially that conversion of the GL starting material did not increase beyond a 90 minute reaction time, and that poor selectivity for GD of only 51% after such time using the standard operating conditions reported therein (0.217 mmol catalyst, 21.73 mmol GL, 65.21 mmol DMC, T=80° 0, t=90 min). Consequently, the authors conducted investigations into the factors affecting conversion rates and selectivity, namely the individual effects of catalyst concentration, reactant ratio and reaction temperature in an attempt to improve selectivity. Insofar as possible, the same standard set of reactions conditions was employed in each case.

The effect of catalyst concentration on activity and selectivity is reported for catalyst loadings of between approximately 0.5 and 4 mol %, based on the amount of GL, at 30 minutes into the standard reaction reported therein. A GL conversion rate of 98% was found to be obtainable with a catalyst loading of 3 mol % (0.651 mmol) after that length of time.

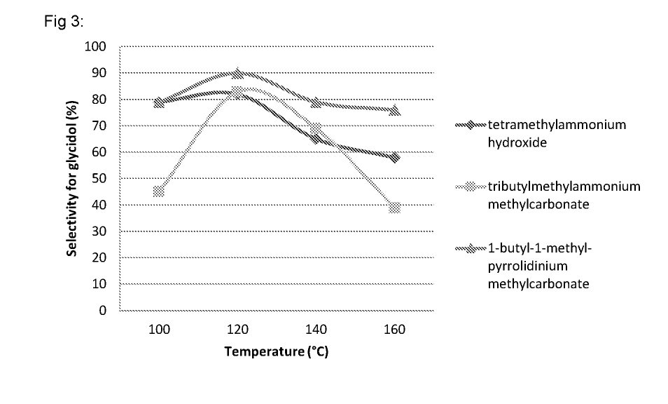

Whilst selectivity for GD was shown to increase on increasing the catalyst loading from approximately 0.5 to 4 mol % (0.108 to 0.868 mmol), it only resulted in a selectivity of 70%, and further increases in catalyst loading were shown to have an insignificant effect on selectivity. The effect of catalyst loading is also shown over the full duration of the standard 90 minute reaction reported therein for catalyst loadings of up to 6 mol % (1.302 mmol), the maximum catalyst loading reported in Gade et al, and demonstrates a levelling-off of GD selectivity, despite further increases in catalyst loading (FIG. 3 of Gade et al).

With respect to reactant ratio, Gade et al reports that a high conversion (97%) was observed for a GL:DMC ratio of 1:3, compared to only 55% at a GL:DMC ratio of 3:1. However, it is reported that GD selectivity was not affected significantly by changes in GL:DMC ratio. Selectivity for these investigations is reported to be only between 43 and 55%, when tested according to the standard operating conditions reported therein. These results are represented in graph format in FIG. 2 of the present application which shows a peak at 2:1 and decreasing selectivity for 3:1.

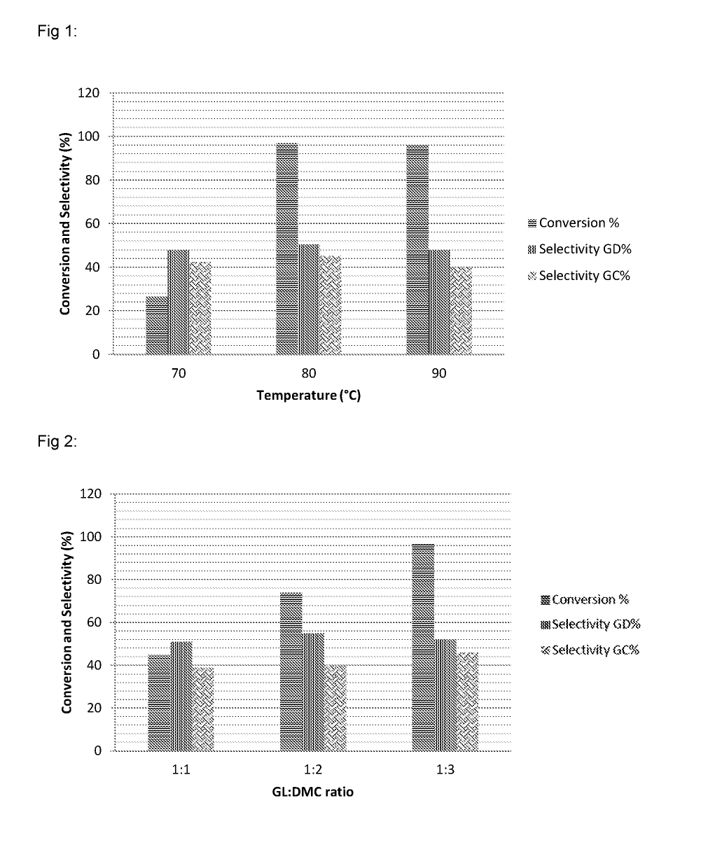

Gade et al also reports the effect of temperature on conversion and selectivity at three different temperatures (70, 80 and 90° C.). Conversion of glycerol was found to increase significantly with increase in temperature from 70 to 80° C. However, no further improvement in conversion was observed on increasing the reaction temperature from 80 to 90° C. These results are represented in graph format in FIG. 1 of the present application.

Further, and of note, changes in reaction temperature were not found to significantly affect GD selectivity, which is consistently shown to be around 50%, and actually decreasing with increasing temperature, when tested according to the standard operating conditions reported in Gade et al.

Although the process reported in Gade et al is an alternative to traditional commercial processes for preparing glycidol, the selectivity of the process remains low.

J. S. Choi et al., Journal of Catalysis, 297, 2013, pages 248 to 255 (hereinafter referred to as “Choi et al”) discusses a process where pre-formed GC (formed using known non-ionic liquid based systems) undergoes decarboxylation in the presence of an ionic liquid catalyst to form GD and carbon dioxide as a by-product.

Choi et al further reports the results of multiple decarboxylations, including investigations into the effect of catalyst loading on the decarboxylation of GC performed at a temperature of 175° C. and a pressure of 2.67 kPa for 45 minutes. The results show that no discernible improvement in either conversion or GD selectivity resulted from increasing the catalyst load above a catalyst/GC molar ratio of 0.0025 up to a value of 0.020 (equivalent to a catalyst loading of 0.25 to 2 mol %, based on GC).

Choi et al further reports the results of an investigation into the effect of temperature on both conversion and GD selectivity in the decarboxylation reaction, at constant pressure (2.67 kPa). The results show that no GC conversion is achieved below 140° C., and a maximum level of conversion is achieved at 175° C. Selectivity for GD is shown to be approximately 70% at a temperature of 165° C. whilst a maximum selectivity of approximately 75% is shown as a result of increasing the temperature to 175° C. However, further increases in temperature only had the effect of decreasing GD selectivity. The effect of reaction time at 175° C. was also investigated in Choi et al, from which it was found that the decarboxylation reaction is completed within 30 minutes. Thus, Choi et al favours higher temperatures (175° C.) than used by Gade et al in the alternative synthesis of GD (70 to 90° C.).

According to Choi et al, the only means for obtaining a GD selectivity of more than 78% in the decarboxylation of GC is to utilize a high-boiling point solvent, to minimise interaction of GD with the ionic liquid catalyst, together with simultaneous removal of GD as soon as it is formed. This is accomplished in Choi et al by performing the reaction at a reduced pressure. The improvement in selectivity is shown to be more pronounced for a continuous rather than batch decarboxylation process utilising the high-boiling point solvent and a maximum selectivity of 98% is reported.

Although superior GD selectivity is reported by Choi et al in comparison with Gade et al, Choi et al relies on the use of GC as a starting material. GC is significantly more expensive than GL and less readily available. Consequently, the use of GC as a starting material is not preferred. Although it would be possible to isolate GC from a transesterification of GL and DMC, this introduces extra steps into the preparation of GD and makes the process less economical.

It therefore remains desirable for there to be a process which is capable of producing GD directly from GL in an efficient one-pot synthesis, with a high GD selectivity and high conversion.

The present invention is based on the surprising discovery that GD selectivity may be enhanced in a one-pot, ionic liquid catalysed synthetic process for the preparation of GD from GL and DMC, whilst maintaining high conversion, by conducting the reaction at a temperature of from 100° C. to 160° C. and using a molar ratio of glycerol to dimethylcarbonate of from 1:4 to 1:10.

In a first aspect, the present invention provides a one-pot synthetic process for the preparation of glycidol comprising the reaction of glycerol and dimethyl carbonate in the presence of an ionic liquid catalyst having the formula:

[Cat⁺][X⁻]
wherein: [Cat⁺] represents one or more cationic species, and
   [X⁻] represents one or more anionic species;
      wherein the reaction is conducted at a temperature of from 100° C. to 160° C. and wherein the molar ratio of glycerol to dimethylcarbonate is from 1:4 to 1:10.

The present invention relates to a particular selection of reaction conditions that have surprisingly been found to be advantageous in terms of glycerol conversion and glycidol selectivity achieved in the synthesis of glycidol from glycerol and dimethyl carbonate. The particular reaction conditions which lead to the surprising benefits are: i) conducting the reaction at a temperature of from 100° C. to 160° C.; ii) conducting the reaction with a GL:DMC molar ratio of from 1:4 to 1:10, for example a GL:DMC ratio of 1:5 or 1:8, in the presence of an ionic liquid catalyst.

It is particularly surprising that the process of the present invention leads to both high conversion and superior selectivity based on the known prior art method for a one-pot synthesis of glycidol from glycerol and dimethyl carbonate using an ionic liquid catalyst. In the method reported in Gade et al, selectivity was not affected significantly by changes in GL:DMC molar ratio. Gade et al reports that increasing either GL or DMC concentration increases conversion. Whilst illustrated by an increase from 45% to 97% GL conversion as a result of changing the GL:DMC molar ratio from 1:1 to 1:3, selectivity was poor. With regard to the investigations into the effect of reactant ratio in Gade et al, the highest GD selectivity (55%) was observed for a GL:DMC molar ratio of 1:2. This reported selectivity is still very low. It is therefore entirely unexpected that the process of the present invention would lead to high conversion as well as high GD selectivity in the light of the information in Gade et al.

In accordance with the present invention, the molar ratio of glycerol to dimethylcarbonate is from 1:4 to 1:10, preferably from 1:5 to 1:8, such as for example 1:6 to 1:7. Thus, exemplary molar ratios of glycerol to dimethylcarbonate include: 1:5, 1:6, 1:7 or 1:8.

The process of the present invention is preferably conducted at a temperature of 110° C. to 140° C., more preferably from 115° C. to 130° C. A temperature of from 115° C. to 125° C., for example 120° C., has been found to be particularly beneficial with the process of the present invention.

Heating may be accomplished using any suitable method, of which those skilled in the art would be readily aware. For example, the reaction may be heated using conventional thermal methods, microwave heating or employing other heat sources such as ultrasound or infrared radiation. In one embodiment of the invention, heating is accomplished by conventional thermal heating. In another embodiment of the invention, heating is accomplished by microwave heating in a microwave reactor.

The inventors have also found that GD selectivity may be enhanced in a one-pot, ionic liquid catalysed synthetic process for the preparation of GD from GL and DMC, whilst maintaining high conversion, by conducting the reaction in a microwave reactor at a temperature of from 100° C. to 160° C.

This process may also benefit from a shorter reaction time for preparing the GD product than conventional non-microwave methods known in the art, whilst being advantageous in terms of glycerol conversion and glycidol selectivity achieved in the synthesis of glycidol from glycerol and dimethyl carbonate.

Thus, in another aspect, the present invention provides a one-pot synthetic process for the preparation of glycidol comprising the reaction of glycerol and dimethyl carbonate in the presence of an ionic liquid catalyst having the formula:

[Cat⁺][X⁻]
wherein: [Cat⁺] represents one or more cationic species, and
   [X⁻] represents one or more anionic species;
      wherein the reaction is conducted in a microwave reactor at a temperature of from 100° C. to 160° C. In this aspect, the molar ratio of glycerol to dimethylcarbonate is preferably from 1:4 to 1:10, more preferably from 1:5 to 1:8, such as for example 1:6 to 1:7. Thus, exemplary molar ratios of glycerol to dimethylcarbonate include: 1:5, 1:6, 1:7 or 1:8. Preferably the process according this aspect of the invention is conducted at a temperature of 110° C. to 140° C., more preferably from 115° C. to 130° C. A temperature of from 115° C. to 125° C., for example 120° C., has been found to be particularly beneficial with the process of the present invention.

In addition, the inventors have also found that GD selectivity may be enhanced in a one-pot, ionic liquid catalysed synthetic process for the preparation of GD from GL and DMC, whilst maintaining high conversion, by conducting the reaction in a microwave reactor using a molar ratio of glycerol to dimethylcarbonate of from 1:4 to 1:10.

This process may also benefit from a shorter reaction time for preparing the GD product than conventional non-microwave methods known in the art, whilst being advantageous in terms of glycerol conversion and glycidol selectivity achieved in the synthesis of glycidol from glycerol and dimethyl carbonate.

Thus, in a further aspect, the present invention provides a one-pot synthetic process for the preparation of glycidol comprising the reaction of glycerol and dimethyl carbonate in the presence of an ionic liquid catalyst having the formula:

[Cat⁺][X⁻]
wherein: [Cat⁺] represents one or more cationic species, and
   [X⁻] represents one or more anionic species;
      wherein the reaction is conducted in a microwave reactor and wherein the molar ratio of glycerol to dimethylcarbonate is from 1:4 to 1:10. In this aspect, the process according this aspect of the invention is preferably conducted at a temperature of from 100° C. to 160° C., more preferably from 110° C. to 140° C., most preferably from 115° C. to 130° C. A temperature of from 115° C. to 125° C., for example 120° C., has been found to be particularly beneficial with the process of the present invention. Preferably, the molar ratio of glycerol to dimethylcarbonate is from 1:5 to 1:8, such as for example 1:6 to 1:7. Thus, exemplary molar ratios of glycerol to dimethylcarbonate include: 1:5, 1:6, 1:7 or 1:8.

Where a microwave reactor apparatus is used with the process of the present invention, heating is provided by microwave energy (i.e. electromagnetic radiation of a frequency of about 10⁸ Hz to 10¹² Hz) generated by a magnetron, typically operating at a frequency of 2450 MHz. The reaction mixture may be heated in open or, preferably, sealed vessels. Preferably, the microwave reactor is automated such that a particular temperature, maximum pressure, maximum power output and hold time can be specified during operation. Suitable microwave reactors for use with the present invention include the CEM Explorer and Anton Paar Monowave 300 microwave reactors.

The process of the present invention may be conducted at a pressure of from 10,000 to 1,500,000 Pa (0.1 to 15 bar), more preferably from 10,000 to 1,000,000 Pa (0.1 to 10 bar), and most preferably 50,000 to 500,000 Pa (0.5 to 5 bar).

As would be understood by those of skill in the art, the ionic liquid and the glycerol and dimethyl carbonate reactants may be reacted by means of continuous processes or batch processes.

Any conventional liquid-liquid or gas-liquid contactor apparatus may be used in accordance with the present invention. For instance, the ionic liquid and the glycerol and dimethyl carbonate reactants may be reacted using a counter-current liquid-liquid contactor, a co-current liquid-liquid contactor, a counter-current gas-liquid contactor, a co-current gas-liquid contactor, a liquid-liquid batch contactor, or a gas-liquid batch contactor.

The term “ionic liquid” as used herein refers to a liquid that is capable of being produced by melting a salt, and when so produced consists solely of ions. An ionic liquid may be formed from a homogeneous substance comprising one species of cation and one species of anion, or it can be composed of more than one species of cation and/or more than one species of anion. Thus, an ionic liquid may be composed of more than one species of cation and one species of anion. An ionic liquid may further be composed of one species of cation, and one or more species of anion. Still further, an ionic liquid may be composed of more than one species of cation and more than one species of anion.

The term “ionic liquid” includes compounds having both high melting points and compounds having low melting points, e.g. at or below room temperature. Thus, many ionic liquids have melting points below 200° C., particularly below 100° C., around room temperature (15 to 30° C.), or even below 0° C. Ionic liquids having melting points below around 30° C. are commonly referred to as “room temperature ionic liquids” and are often derived from organic salts having nitrogen-containing heterocyclic cations. In room temperature ionic liquids, the structures of the cation and anion prevent the formation of an ordered crystalline structure and therefore the salt is liquid at room temperature.

Ionic liquids are most widely known as solvents. Many ionic liquids have been shown to have negligible vapour pressure, temperature stability, low flammability and recyclability. Due to the vast number of anion/cation combinations that are available it is possible to fine-tune the physical properties of the ionic liquid (e.g. melting point, density, viscosity, and miscibility with water or organic solvents) to suit the requirements of a particular application.

The term “catalyst” as used herein refers to a substance which increases the rate of a chemical reaction without itself being consumed by the reaction. In particular, the ionic liquid catalyst used increases the rate of transesterification between glycerol and dimethylcarbonate to form glycerol carbonate and/or increases the rate of decarboxylation of glycerol carbonate to form glycidol.

In accordance with the present invention, [Cat⁺] may comprise a cationic species selected from: ammonium, benzimidazolium, benzofuranium, benzothiophenium, benzotriazolium, borolium, cinnolinium, diazabicyclodecenium, diazabicyclononenium, 1,4-diazabicyclo[2.2.2]octanium, diazabicyclo-undecenium, dithiazolium, furanium, guanidinium, imidazolium, indazolium, indolinium, indolium, morpholinium, oxaborolium, oxaphospholium, oxazinium, oxazolium, iso-oxazolium, oxothiazolium, phospholium, phosphonium, phthalazinium, piperazinium, piperidinium, pyranium, pyrazinium, pyrazolium, pyridazinium, pyridinium, pyrimidinium, pyrrolidinium, pyrrolium, quinazolinium, quinolinium, iso-quinolinium, quinoxalinium, quinuclidinium, selenazolium, sulfonium, tetrazolium, thiadiazolium, iso-thiadiazolium, thiazinium, thiazolium, iso-thiazolium, thiophenium, thiuronium, triazinium, triazolium, iso-triazolium, and uronium.

In one preferred embodiment of the invention, [Cat⁺] comprises an acyclic cation selected from:

[N(R^{a})(R^{b})(R^{c})(R^{d})]⁺,[P(R^{a})(R^{b})(R^{c})(R^{d})]⁺, and [S(R^{a})(R^{b})(R^{c})]⁺,
wherein: R^{a}, R^{b}, R^{c}, and R^{d} are each independently selected from a C₁ to C₃₀, straight chain or branched alkyl group, a C₃ to C₈ cycloalkyl group, or a C₆ to C₁₀ aryl group; and wherein said alkyl, cycloalkyl or aryl groups are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₋₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

More preferably, [Cat⁺] comprises a cation selected from:

[N(R^{a})(R^{b})(R^{c})(R^{d})]⁺,[P(R^{a})(R^{b})(R^{c})(R^{d})]⁺, and [S(R^{a})(R^{b})(R^{c})]⁺,
wherein: R^{a}, R^{b}, R^{c}, and R^{d} are each independently selected from a C₁ to C₁₅ straight chain or branched alkyl group, a C₃ to C₆ cycloalkyl group, or a C₆ aryl group; and wherein said alkyl, cycloalkyl or aryl groups are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆ alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

Further examples include wherein R^{a}, R^{b}, R^{c} and R^{d} are independently selected from methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl and n-octadecyl.

More preferably two or more, and most preferably three or more, of R^{a}, R^{b}, R^{c} and R^{d} are selected from methyl, ethyl, propyl and butyl.

Still more preferably, [Cat⁺] comprises a cation selected from:

[N(R^{a})(R^{b}(R^{c})(R^{d})]⁺,
wherein: R^{a}, R^{b}, R^{c}, and R^{d} are as defined above.

In a preferred further embodiment, [Cat⁺] preferably comprises a cation selected from:

[P(R^{a})(R^{b})(R^{c})(R^{d})]⁺,
wherein: R^{a}, R^{b}, R^{c}, and R^{d} are as defined above.

Specific examples of preferred ammonium and phosphonium cations suitable for use according to the present invention include:

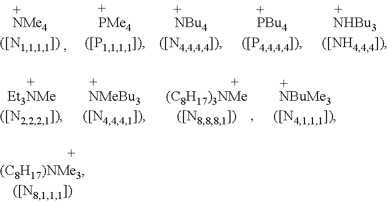

Specific examples of more preferred ammonium cations suitable for use according to the present invention include:

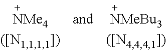

In a further embodiment of the invention, [Cat⁺] comprises a cation selected from guanidinium, cyclic guanidinium, uronium, cyclic uronium, thiuronium and cyclic thiuronium.

More preferably, [Cat⁺] comprises a cation having the formula:

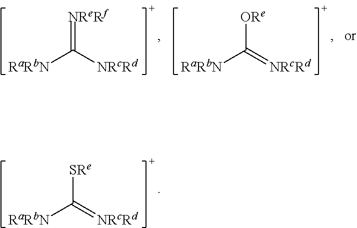

wherein: R^{a}, R^{b}, R^{c}, R^{d}, R^{e}, and R^{f} are each independently selected from a C₁ to C₃₀, straight chain or branched alkyl group, a C₃ to C₈ cycloalkyl group, or a C₆ to C₁₀ aryl group, or any two of R^{a}, R^{b}, R^{c}, and R^{d}, attached to different nitrogen atoms form a methylene chain —(CH₂)_{q}— wherein q is from 2 to 5; wherein said alkyl, cycloalkyl or aryl groups or said methylene chain are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(c, to C₆)alkyl, —OS(O)(C₁ to C₆)alkyl, —S(C₁ to C₆)alkyl, —S—S(C₁ to C₆ alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

Specific examples of guanidinium, uronium, and thiuronium cations suitable for use according to the present invention include:

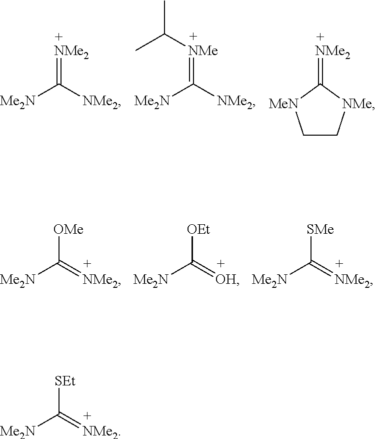

In a further preferred embodiment, [Cat⁺] comprises a cation comprising an electron-rich sulfur or selenium moiety. Examples include cations as defined above comprising pendant thiol, thioether, or disulfide substituents.

In another embodiment of the invention, [Cat⁺] comprises an aromatic heterocyclic cationic species selected from: benzimidazolium, benzofuranium, benzothiophenium, benzotriazolium, cinnolinium, diazabicyclodecenium, diazabicyclononenium, diazabicyclo-undecenium, dithiazolium, imidazolium, indazolium, indolinium, indolium, oxazinium, oxazolium, iso-oxazolium, oxathiazolium, phthalazinium, pyrazinium, pyrazolium, pyridazinium, pyridinium, pyrimidinium, quinazolinium, quinolinium, iso-quinolinium, quinoxalinium, tetrazolium, thiadiazolium, iso-thiadiazolium, thiazinium, thiazolium, iso-thiazolium, triazinium, triazolium, and iso-triazolium.

More preferably, [Cat⁺] has the formula:

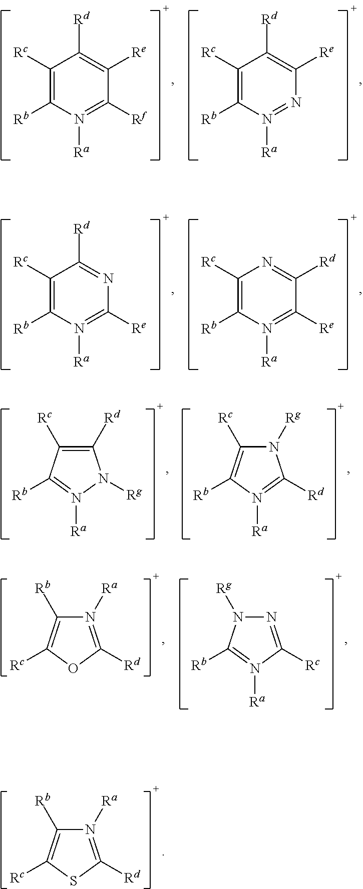

wherein: R^{a}, R^{b}, R^{c}, R^{d}, R^{e}, R^{f} and R^{g} are each independently selected from hydrogen, a C₁ to C₃₀, straight chain or branched alkyl group, a C₃ to C₈ cycloalkyl group, or a C₆ to C₁₀ aryl group, or any two of R^{b}, R^{c}, R^{d}, R^{e} and R^{f} attached to adjacent carbon atoms form a methylene chain —(CH₂)_{q}— wherein q is from 3 to 6; and wherein said alkyl, cycloalkyl or aryl groups or said methylene chain are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆ alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S) NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

R^{a} is preferably selected from C₁ to C₃₀, linear or branched, alkyl, more preferably C₂ to C₂₀ linear or branched alkyl, still more preferably, C₂ to C₁₀ linear or branched alkyl, and most preferably C₄ to C₈ linear or branched alkyl. Further examples include wherein R^{a} is selected from methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl and n-octadecyl.

In the cations comprising an R^{g} group, R^{g} is preferably selected from C₁ to C₁₀ linear or branched alkyl, more preferably, C₁ to C₅ linear or branched alkyl, and most preferably R^{g} is a methyl group.

In the cations comprising both an R^{a} and an R^{g} group, R^{a} and R^{g} are each preferably independently selected from C₁ to C₃₀, linear or branched, alkyl, and one of R^{a} and R^{g} may also be hydrogen. More preferably, one of R^{a} and R^{g} may be selected from C₂ to C₂₀ linear or branched alkyl, still more preferably, C₂ to C₁₀ linear or branched alkyl, and most preferably C₄ to C₈ linear or branched alkyl, and the other one of R^{a} and R^{g} may be selected from C₁ to C₁₀ linear or branched alkyl, more preferably, C₁ to C₅ linear or branched alkyl, and most preferably a methyl group. In a further preferred embodiment, R^{a} and R^{g} may each be independently selected, where present, from C₁ to C₃₀ linear or branched alkyl and C₁ to C₁₅ alkoxyalkyl.

In further preferred embodiments, R^{b}, R^{c}, R^{d}, R^{e}, and R^{f} are independently selected from hydrogen and C₁ to C₅ linear or branched alkyl, and more preferably R^{b}, R^{c}, R^{d}, R^{e}, and R^{f} are hydrogen.

In this embodiment of the invention, [Cat⁺] preferably comprises a cation selected from:

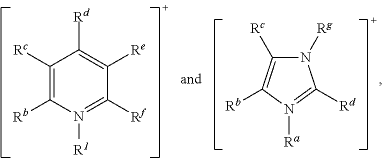

wherein: R^{a}, R^{b}, R^{c}, R^{d}, R^{e}, R^{f}, and R^{g} are as defined above.

More preferably, [Cat⁺] comprises a cation selected from:

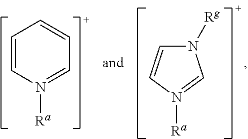

wherein: R^{a} and R^{g} are as defined above.

Also in accordance with this embodiment of the invention, [Cat⁺] may preferably comprise a cation selected from:

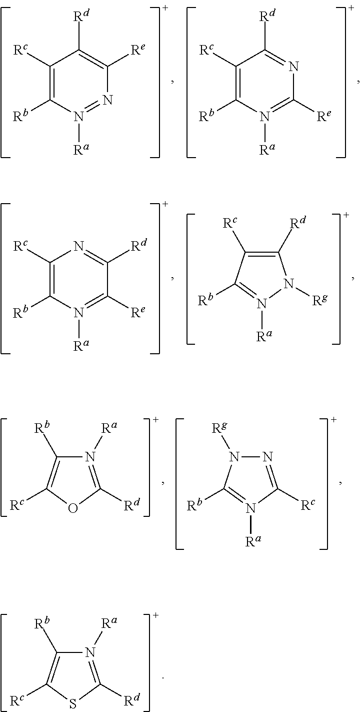

wherein: R^{a}, R^{b}, R^{c}, R^{d}, R^{e}, R^{f} and R^{g} are as defined above.

Specific examples of preferred nitrogen-containing aromatic heterocyclic cations that may be used according to the present invention include:

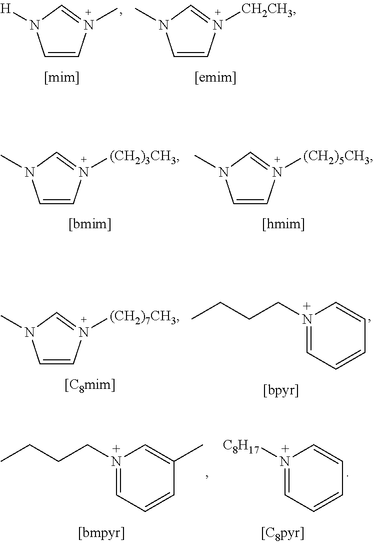

In another preferred embodiment of the invention, [Cat⁺] comprises a saturated heterocyclic cation selected from cyclic ammonium, 1,4-diazabicyclo[2.2.2]octanium, morpholinium, cyclic phosphonium, piperazinium, piperidinium, quinuclidinium, and cyclic sulfonium.

More preferably, [Cat⁺] comprises a saturated heterocyclic cation having the formula:

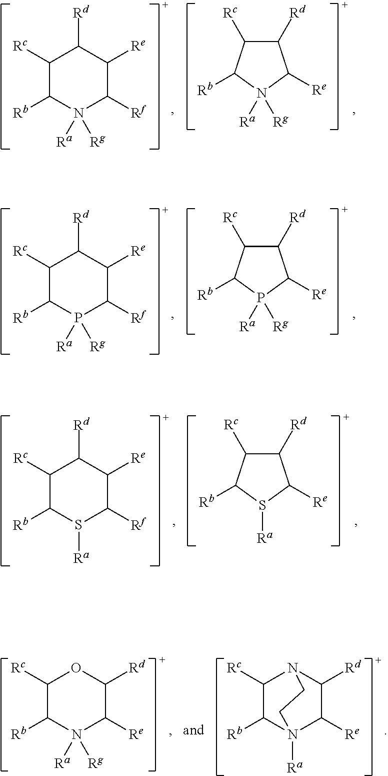

wherein: R^{a}, R^{b}, R^{c}, R^{d}, R^{e}, R^{f}, and R^{g} are as defined above.

Still more preferably, [Cat⁺] comprises a saturated heterocyclic cation having the formula:

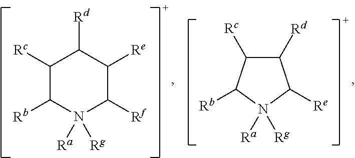

and is most preferably

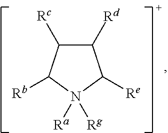

wherein: R^{a}, R^{b}, R^{c}, R^{d}, R^{e}, R^{f}, and R^{g} are as defined above.

A specific example of a preferred saturated heterocyclic cation suitable for use according to the present invention is 1-butyl-1-methylpyrrolidinium cation:

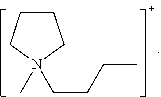

Also in accordance with this embodiment of the invention, [Cat⁺] may preferably comprise a saturated heterocyclic cation selected from:

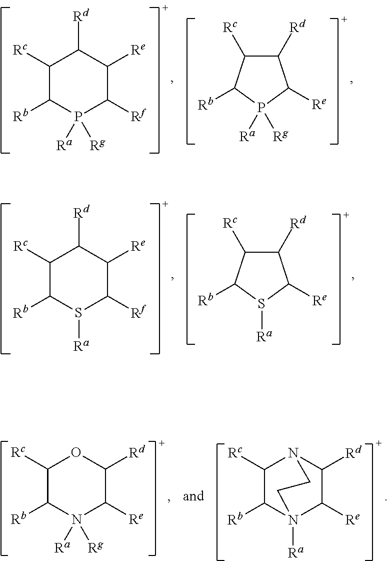

wherein: R^{a}, R^{b}, R^{c}, R^{d}, R^{e}, R^{f}, and R^{g} are as defined above.

In the saturated heterocyclic cations above, R^{a} is preferably selected from C₁ to C₃₀, linear or branched, alkyl, more preferably C₂ to C₂₀ linear or branched alkyl, still more preferably, C₂ to C₁₀ linear or branched alkyl, and most preferably C₄ to C₈ linear or branched alkyl. Further examples include wherein R^{a} is selected from methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl and n-octadecyl.

In the saturated heterocyclic cations comprising an R^{g} group, R^{g} is preferably selected from C₁ to C₁₀ linear or branched alkyl, more preferably, C₁ to C₅ linear or branched alkyl, and most preferably R^{g} is a methyl group.

In the saturated heterocyclic cations comprising both an R^{a} and an R^{g} group, R^{a} and R^{g} are each preferably independently selected from C₁ to C₃₀, linear or branched, alkyl, and one of R^{a} and R^{g} may also be hydrogen. More preferably, one of R^{a} and R^{g} may be selected from C₂ to C₂₀ linear or branched alkyl, still more preferably, C₂ to C₁₀ linear or branched alkyl, and most preferably C₄ to C₈ linear or branched alkyl, and the other one of R^{a} and R^{g} may be selected from C₁ to C₁₀ linear or branched alkyl, more preferably, C₁ to C₅ linear or branched alkyl, and most preferably a methyl group. In a further preferred embodiment, R^{a} and R^{g} may each be independently selected, where present, from C₁ to C₃₀ linear or branched alkyl and C₁ to C₁₅ alkoxyalkyl.

In further preferred embodiments, R^{b}, R^{c}, R^{d}, R^{e}, and R^{f} are independently selected from hydrogen and C₁ to C₅ linear or branched alkyl, and more preferably R^{b}, R^{c}, R^{d}, R^{e}, and R^{f} are hydrogen.

In accordance with the present invention, [X⁻] may comprise one or more anions selected from hydroxides, halides, perhalides, pseudohalides, sulphates, sulphites, sulfonates, sulfonimides, phosphates, phosphites, phosphonates, methides, borates, carboxylates, azolates, carbonates, carbamates, thiophosphates, thiocarboxylates, thiocarbamates, thiocarbonates, xanthates, thiosulfonates, thiosulfates, nitrate, nitrite, perchlorate, halometallates, amino acids and borates.

Thus, [X⁻] may represent one or more anions selected from:
a) a halide anion selected from: F⁻, Cl⁻, Br⁻, I⁻;
b) a perhalide anion selected from: [I₃]⁻, [I₂Br]⁻, [IBr₂]⁻, [Br₃]⁻, [Br₂C]⁻, [BrCl₂]⁻, [ICl₂]⁻, [I₂Cl]⁻, [Cl₃]⁻;
c) a pseudohalide anion selected from: [N₃]⁻, [NCS]⁻, [NCSe]⁻, [NCO]⁻, [CN]⁻;
d) a sulphate anion selected from: [HSO₄]⁻, [SO₄]²⁻, [R²OSO₂O]⁻;
e) a sulphite anion selected from: [HSO₃]⁻, [SO₃]²⁻, [R²OSO₂]⁻;
f) a sulfonate anion selected from: [R¹SO₂O]⁻;
g) a sulfonimide anion selected from: [(R¹SO₂)₂N]⁻;
h) a phosphate anion selected from: [H₂PO₄]⁻, [HPO₄]²⁻, [PO₄]³⁻, [R²OPO₃]²⁻, [(R²O)₂PO₂]⁻,
i) a phosphite anion selected from: [H₂PO₃]⁻, [HPO₃]²⁻, [R²OPO₂]²⁻, [(R²O)₂PO]⁻;
j) a phosphonate anion selected from: [R¹PO₃]²⁻, [R¹P(O)(OR²)O]⁻;
k) a methide anion selected from: [(R¹SO₂)₃C]⁻;
l) a borate anion selected from: [bisoxalatoborate], [bismalonatoborate];
m) a carboxylate anion selected from: [R²CO₂]⁻;
n) an azolate anion selected from: [3,5-dinitro-1,2,4-triazolate], [4-nitro-1,2,3-triazolate], [2,4-dinitroimidazolate], [4,5-dinitroimidazolate], [4,5-dicyano-imidazolate], [4-nitroimidazolate], [tetrazolate];
o) a sulfur-containing anion selected from: thiocarbonates (e.g. [R²OCS₂]⁻); thiocarbamates and (e.g. [R²₂NCS₂]⁻); thiocarboxylates (e.g. [R¹CS₂]⁻); thiophosphates (e.g. [(R²O)₂PS₂]⁻); thiosulfonates (e.g. [RS(O)₂S]⁻); and thiosulfates (e.g. [ROS(O)₂S]⁻); and
p) a nitrate ([NO₃]⁻) or nitrite ([NO₂]⁻) anion;
q) a carbonate anion selected from [CO₃]²⁻, [HCO₃]⁻, [R²CO₃]⁻; preferably [MeCO₃]⁻;
wherein: R¹ and R² are independently selected from the group consisting of C₁-C₁₀ alkyl, C₆ aryl, C₁-C₁₀ alkyl(C₆)aryl, and C₆ aryl(C₁-C₁₀)alkyl each of which may be substituted by one or more groups selected from: fluoro, chloro, bromo, iodo, C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₋₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆ alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl, and wherein R¹ may also be fluorine, chlorine, bromine or iodine.

In one preferred embodiment, [X⁻] comprises a halide or perhalide anion selected from: [F]⁻, [Cl]⁻, [Br]⁻, [I]⁻, [I₃]⁻, [I₂Br]⁻, [IBr₂]⁻, [Br₃]⁻, [Br₂Cl]⁻, [BrCl₂]⁻, [ICl₂]⁻, [I₂Cl]⁻, [Cl₃]⁻. More preferably [X⁻] comprises a halide or perhalide anion selected from: [F]⁻, [Cl]⁻, [Br]⁻, [I]⁻, [I₂Br]⁻, [IBr₂]⁻, [Br₂Cl]⁻, [BrCl₂]⁻, [ICl₂]⁻, [I₂Cl]⁻.

In a further preferred embodiment, [X⁻] comprises an oxygen-containing anion selected from: [NO₃]⁻, [NO₂]⁻, [H₂PO₄]⁻, [HPO₄]²⁻, [PO₄]³⁻, [R²OPO₃]²⁻, [(R²O)₂PO₂]⁻, [H₂PO₃]⁻, [HPO₃]²⁻, [R²OPO₂]²⁻, [(R²O)₂PO]⁻, [R¹PO₃]²⁻, [R¹P(O)(OR²)O]⁻, wherein R¹ and R² are as defined above. Further examples of anions in this category include: [MeOPO₃]²⁻, [EtOPO₃]²⁻, [(MeO)₂PO₂]⁻, [(EtO)₂PO₂]⁻, [MePO₃]²⁻, [EtPO₃]²⁻, [MeP(O)(OMe)O]⁻, [EtP(O)(OEt)O]⁻.

In a further preferred embodiment, [X⁻] comprises a carboxylate anion selected from [R²CO₂]⁻; wherein R² is as defined above. Further examples of anions in this category include: [HCO₂]⁻, [MeCO₂]⁻, [EtCO₂]⁻, [CH₂(OH)CO₂]⁻, [CH₃CH(OH)CH₂CO₂]⁻, [PhCO₂]⁻, salicylate, alaninate, argininate, asparaginate, aspartate, cysteinate, glutamate, glutaminate, glycinate, histidinate, isoleucinate, leucinate, lysinate, methioninate, phenylalaninate, prolinate, serinate, threoninate, tryptophanate, tyrosinate, valinate, N-methylglycinate, 2-aminobutyrate, 2-aminoisobutyrate, 2-amino-4-aminooxy-butyrate, 2-(methylguanidino)-ethanoate, 2-pyrrolidone-5-carboxylate, piperidine-2-carboxylate, and 1-piperidinepropionate.

In a further preferred embodiment, [X⁻] comprises an anion comprising an electron-rich sulfur or selenium moiety. Examples include: anions as defined above comprising pendant thiol, thioether, or disulfide substituents, [NCS]⁻, [NCSe]⁻, [R²OCS₂]⁻, [R²₂NCS₂]⁻, [R¹CS₂]⁻, [(R²O)₂PS₂]⁻, [R¹S(O)₂S]⁻ and [R²OS(O)₂S]⁻, wherein R¹ and R² are as defined above. Further examples of anions in this category include: [CH₂(SH)CO₂]⁻, [CH₃CH₂(SH)CO₂]⁻, [CH₃CS₂]⁻, [CH₃CH₂CS₂]⁻, [PhCS₂]⁻, [(MeO)₂PS₂]⁻, [(EtO)₂PS₂]⁻, [(PhO)₂PS₂]⁻, [(CH₃)₂NCS₂]⁻, [(CH₃CH₂)₂NCS₂]⁻, [Ph₂NCS₂]⁻, [CH₃OCS₂]⁻, [CH₃CH₂OCS₂]⁻, [PhOCS₂]⁻,

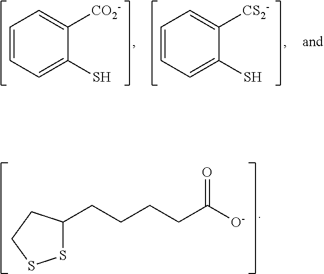

In a further preferred embodiment, [X⁻] comprises a sulfur-containing anion selected from sulphate anions ([HSO₄]⁻, [SO₄]²⁻, [R²OSO₂O]), sulphite anions ([HSO₃]⁻, [SO₃]²⁻, [R²OSO₂]⁻) and sulfonate anions ([R¹SO₂O]⁻). Further examples of anions in this category include: [FSO₂O]⁻, [CF₃SO₂O]⁻, [MeSO₂O]⁻, [PhSO₂O]⁻, [4-MeC₆H₄SO₂O]⁻, [dioctylsulfosuccinate]⁻, [MeOSO₂O]⁻, [EtOSO₂O]⁻, [C₈H₁₇OSO₂O]⁻, and [MeOSO₂]⁻, [PhOSO₂]⁻.

In a further preferred embodiment, [X⁻] comprises a carbonate anion selected from [R²CO₃]⁻; wherein R² is defined as above. Preferably, where [X⁻] comprises a carbonate anion selected from selected from [R²CO₃]⁻, R² is selected from methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl and n-octadecyl. More preferably R² is selected from methyl, ethyl, n-propyl, n-butyl, and most preferably R² is methyl.

In a further preferred embodiment, [X⁻] may comprise an anion selected from [OH]⁻ and [SH]⁻.

In a particularly preferred embodiment of the invention, [X⁻] may comprise an anion selected from [CO₃]²⁻, [HCO₃]⁻, [MeCO₃]⁻, [OH]⁻, and [SH]⁻, most preferably an anion selected from [MeCO₃]⁻ and [OH]⁻.

In a further embodiment of the invention, [X⁻] may comprise a fluorinated anion selected from: [BF₄], [CF₃BF₃]⁻, [CF₃CF₂BF₃]⁻, [PF₆]⁻, [CF₃PF₅]⁻, [CF₃CF₂PF₅]⁻, [(CF₃CF₂)₂PF₄]⁻; and [(CF₃CF₂)₃PF₃]⁻. However, fluorinated anions of this type are generally less preferred in comparison with the anion types disclosed above.

The present invention is not limited to ionic liquids comprising anions and cations having only a single charge. Thus, the formula [Cat⁺][X⁻] is intended to encompass ionic liquids comprising, for example, doubly, triply and quadruply charged anions and/or cations. The relative stoichiometric amounts of [Cat⁺] and [X⁻] in the ionic liquid are therefore not fixed, but can be varied to take account of cations and anions with multiple charges. For example, the formula [Cat⁺][X⁻] should be understood to include ionic liquids having the formulae [Cat⁺]₂[X²⁻]; [Cat²⁺][X⁻]₂; [Cat²⁺][X²⁻]; [Cat⁺]₃[X³⁻]; [Cat³⁺][X⁻]₃ and so on.

It will also be appreciated that the present invention is not limited to ionic liquids comprising a single cation and a single anion. Thus, [Cat⁺] may, in certain embodiments, represent two or more cations, such as a statistical mixture of 1,3-dimethylimidazolium, 1-ethyl-3-methylimidazolium and 1-3-diethylimidazolium. Similarly, [X⁻] may, in certain embodiments, represent two or more anions, such as a mixture of tribromide ([Br₃]⁻) and bistriflimide ([N(SO₂CF₃)₂]⁻).

In one embodiment of the invention, the ionic liquid used in the process of the present invention is tributylmethylammonium methylcarbonate.

In another embodiment of the invention, the ionic liquid used in the process of the present invention is 1-butyl-1-methylpyrrolidinium methylcarbonate.

In a further embodiment of the invention, the ionic liquid used in the process of the present invention is tetramethylammonium hydroxide.

Ionic liquids for use according to the present invention preferably have a melting point of 250° C. or less, more preferably 150° C. or less, still more preferably 100° C. or less, still more preferably 80° C. or less, and most preferably, the ionic liquid has a melting point below 30° C. However, any compound that meets the criteria of being a salt (consisting of a cation and an anion) and which is liquid at the operating temperature and pressure of the process, or exists in a fluid state during any stage of the reaction, may be defined as an ionic liquid for the purposes of the present invention.

It is well known in the art that the properties of ionic liquids may be ‘tuned’ by altering the nature of the cations and the anions. In particular, in the process of the invention, the structure of the cation or cations may be selected so as to obtain an ionic liquid having desired rheological and physical properties, such as liquid range, melting point, viscosity, hydrophobicity and lipophilicity. The selection of suitable cations to obtain ionic liquids having specific properties is well established in the art, and can readily be undertaken by a skilled person.

If desired, the reaction may be conducted in the presence of a solvent which is compatible with the ionic liquid, glycerol, dimethyl ether, glycerol carbonate and glycidol product. The use of a solvent may be appropriate where it is desired to modify the viscosity of an ionic liquid. Suitable solvents for this purpose are non-basic aprotic polar solvents, such as acetonitrile, dimethylsulfoxide, dimethylformamide and sulfolane (tetrahydrothiophene 1,1-dioxide). In one embodiment of the invention, solvent is present in an amount less than 30 wt %, based on the total weight of the reaction mixture. In a further embodiment of the invention, solvent is present in an amount less than 20 wt %, based on the total weight of the reaction mixture.

In another embodiment of the invention, solvent is present in an amount less than 10 wt %, based on the total weight of the reaction mixture. In a further embodiment, the reaction is conducted substantially in the absence of a solvent (i.e. less than 10 wt %, preferably less than 5 wt %, for example 2 wt %, 1 wt % or 0 wt %).

The ionic liquid may be supported on a solid, preferably porous, carrier material which is compatible the process of the present invention. Suitable solid carriers for use in this embodiment of the invention include silica alumina, silica-alumina, and activated carbon. In general, supported ionic liquids for use according to this embodiment of the invention comprise from 50% by weight to 1% by weight of ionic liquid, and more preferably 20% by weight to 1% by weight of ionic liquid.

The amount of ionic liquid catalyst used in the process of the invention is not particularly limited and the skilled person is able to readily identify a suitable amount based on the amount of the reactants.

The ionic liquid catalyst may be present in an amount corresponding to at least 2 mol % based on glycerol, more preferably at least 5 mol %, for example 8 mol % or 10 mol %.

It has been surprisingly found that an amount of at least 3 mol % ionic liquid catalyst based on glycerol is particularly beneficial with the process of the present invention. In one preferred embodiment, the amount of ionic liquid catalyst is at least 8 mol %, even more preferably at least 10 mol % based on glycerol.

In another embodiment of the invention, the ionic liquid is recycled after use in the reaction. Separation of the ionic liquid from product/by-product materials can readily be undertaken by a skilled person using known separation techniques, such as partitioning between different liquid phases (e.g. aqueous and organic liquid phases). Alternatively, advantage may be taken of the negligible vapour pressure of ionic liquids by separation of product/by-product materials into a vapour phase.

The process of the invention is conducted over a suitable timescale to obtain quantitative or near quantitative (e.g. greater than 95%) conversion of glycerol. It will be appreciated that the rate of reaction will vary according to the ionic liquid that is used. In addition, other reaction parameters such as temperature, pressure and the choice of solvent, if any, may also influence the reaction rate. Quantitative or near quantitative conversion of glycerol is preferably obtained following a reaction time of up to 90 minutes, more preferably up to 60 minutes, still more preferably up to 30 minutes and most preferably up to 15 minutes.

Where microwave heating is used, the ionic liquids may be readily integrated into a microwave reaction due to their high microwave absorption capabilities and therefore can support a fast and clean process. Where heating of the reaction is accomplished in a microwave, quantitative or near quantitative conversion of glycerol is preferably obtained following a microwave reaction hold time of up to 90 minutes, more preferably up to 60 minutes, still more preferably up to 30 minutes and most preferably up to 15 minutes. Reference to “hold time” herein means the time a reaction mixture is held in a microwave reactor at a predetermined temperature, and not the total irradiation time of the reaction mixture.

Embodiments of the invention described hereinbefore may be combined with any other compatible embodiments to form further embodiments of the invention. Thus, embodiments relating to temperature of reaction, form of heating, amount of catalyst, GL:DMC ratio and amount of solvent described hereinbefore can be combined in any manner.

For instance, in one preferred embodiment, heating is accomplished by microwave heating in a microwave reactor and the ionic liquid catalyst is present in amount of at least 10 mol % based on glycerol. In another preferred embodiment, heating is accomplished by conventional thermal methods and the ionic liquid catalyst is present in amount of at least 10 mol % based on glycerol. In a further preferred embodiment, the GL:DMC molar ratio is from 1:5 to 1:8 and the ionic liquid catalyst is present in amount of at least 10 mol % based on glycerol. In yet another preferred embodiment, the process is conducted at a temperature of from 115° C. to 125° C., the GL:DMC ratio is from 1:5 to 1:8 and the reaction is conducted in the presence of less than 5 wt % solvent, for example 2 wt %, 1 wt % or 0 wt %. In a particularly preferred embodiment, the process is conducted at a temperature of from 115° C. to 125° C., the GL:DMC molar ratio is from 1:5 to 1:8, the amount of ionic liquid catalyst is at least 10 mol % based on glycerol and the reaction is conducted in the presence of less than 5 wt % solvent, for example 2 wt %, 1 wt % or 0 wt %.

In another aspect, the present invention provides a one-pot synthetic process for the preparation of glycidol comprising the reaction of glycerol and dimethyl carbonate in the presence of an ionic liquid catalyst having the formula:

[Cat⁺][X⁻]
wherein: [Cat⁺] represents one or more cationic species, and
   [X⁻] represents one or more anionic species;
      wherein the reaction is conducted at a temperature of from 100° C. to 160° C. and wherein the ionic liquid catalyst is present in an amount of at least 8 mol % based on glycerol. Preferably, the reaction is conducted at a temperature of from 110° C. to 140° C., and more preferably from 115° C. to 130° C., most preferably 115° C. to 125° C., for example 120° C. Preferably, the ionic liquid catalyst is present in an amount of at least 10 mol % based on glycerol.

Preferably, the molar ratio of glycerol to dimethylcarbonate is from 1:4 to 1:10, more preferably from 1:5 to 1:8, such as for example 1:6 to 1:7. Thus, exemplary molar ratios of glycerol to dimethylcarbonate include: 1:5, 1:6, 1:7 or 1:8.

In a further aspect, the present invention provides a one-pot synthetic process for the preparation of glycidol comprising the reaction of glycerol and dimethyl carbonate in the presence of an ionic liquid catalyst having the formula:

[Cat⁺][X⁻]
wherein: [Cat⁺] represents one or more cationic species, and
   [X⁻] represents one or more anionic species;
      wherein the reaction is conducted in a microwave reactor and wherein the ionic liquid catalyst is present in an amount of at least 8 mol % based on glycerol. Preferably, the reaction is conducted at a temperature of from 100° C. to 160° C., more preferably 110° C. to 140° C., and still more preferably from 115° C. to 130° C., most preferably from 115° C. to 125° C., for example 120° C. Preferably, the ionic liquid catalyst is present in an amount of at least mol % based on glycerol. Preferably, the molar ratio of glycerol to dimethylcarbonate is from 1:4 to 1:10 or less, more preferably from 1:5 to 1:8, such as for example 1:6 to 1:7. Thus, exemplary molar ratios of glycerol to dimethylcarbonate include: 1:5, 1:6, 1:7 or 1:8.

In yet a further aspect, the present invention provides a one-pot synthetic process for the preparation of glycidol comprising the reaction of glycerol and dimethyl carbonate in the presence of an ionic liquid catalyst having the formula:

[Cat⁺][X⁻]
wherein: [Cat⁺] represents one or more cationic species, and
   [X⁻] represents one or more anionic species;
      wherein the ionic liquid catalyst is present in amount of at least 8 mol % based on glycerol and the molar ratio of glycerol to dimethylcarbonate is from 1:4 to 1:10, preferably from 1:5 to 1:8, such as for example 1:6 to 1:7. Thus, exemplary molar ratios of glycerol to dimethylcarbonate include: 1:5, 1:6, 1:7 or 1:8. Preferably, the reaction is conducted at a temperature of from 100° C. to 160° C., more preferably 110° C. to 140° C., and still more preferably from 115° C. to 130° C., most preferably 115° C. to 125° C., for example 120° C. Preferably, the ionic liquid catalyst is present in an amount of at least 10 mol % based on glycerol.

In yet another aspect, the invention provides a process for the preparation of glycidol comprising decarboxylation of glycerol carbonate in the presence of an ionic liquid catalyst having the formula:

[Cat⁺][X⁻]
wherein: [Cat⁺] represents one or more cationic species, and
   [X⁻] represents one or more anionic species;
      wherein the reaction is conducted at a temperature of from 100° C. to 160° C.; and wherein the amount of ionic liquid catalyst is at least 3 mol % based on glycerol carbonate. Preferably, the reaction is conducted at a temperature of from 110° C. to 140° C., and more preferably from 115° C. to 130° C., most preferably 115° C. to 125° C., for example 120° C. Preferably, the ionic liquid catalyst is present in an amount of at least 5 mol % based on glycerol carbonate, more preferably at least 8 mol % and most preferably the ionic liquid catalyst is present in an amount corresponding to at least 10 mol % based on glycerol carbonate.

In the above additional aspects of the invention, the ionic liquid may be selected from any of the ionic liquids described hereinbefore, or formed from any combination of cationic species ([Cat⁺]) and anionic species ([X⁻]) described hereinbefore. Thus, for example, the ionic liquid used is tributylmethylammonium methylcarbonate. In another embodiment of the invention, the ionic liquid used is 1-butyl-1-methylpyrrolidinium methylcarbonate. In a further embodiment of the invention, the ionic liquid used is tetramethylammonium hydroxide. Furthermore, in the above additional aspects of the invention, the ionic liquid may be fixed onto a solid support or recycled as described hereinbefore.

If desired, the reaction according to the above additional aspects of the invention may be conducted in the presence of a solvent which is compatible with the ionic liquid, glycerol/dimethyl carbonate and/or glycerol carbonate and glycidol product, as described hereinbefore for other embodiments of the invention. Thus, in the above additional aspects of the invention, solvent is present in an amount less than 30 wt %, based on the total weight of the reaction mixture. In a further embodiment of the invention, solvent is present in an amount less than 20 wt %, based on the total weight of the reaction mixture. In another embodiment of the invention, solvent is present in an amount less than 10 wt %, based on the total weight of the reaction mixture. In a further embodiment, the reaction according to any of the additional aspects of the invention is conducted substantially in the absence of a solvent (i.e. less than 10 wt %, preferably less than 5 wt %, for example 2 wt %, 1 wt % or 0 wt %).

Unless specified particularly, heating of the reaction according to the above additional aspects of the invention may be accomplished by any suitable means, including those described hereinbefore. Thus, unless otherwise specified, in one embodiment of the invention, heating is accomplished by conventional thermal heating. In another embodiment of the invention, heating is accomplished by microwave heating in a microwave reactor. Reaction timescales and hold times described hereinbefore also apply equally to the above additional aspects of the invention.

Embodiments relating to the above additional aspects of the invention may be combined with any other compatible embodiments to form yet further embodiments of the invention. Thus, embodiments relating to temperature of reaction, form of heating, amount of catalyst, GL:DMC molar ratio (if relevant) and amount of solvent described hereinbefore can be combined in any manner.

For instance, in the additional aspect directed to a process for preparing glycidol from decarboxylation of glycerol carbonate, in one preferred embodiment, heating is accomplished by microwave heating in a microwave reactor and the ionic liquid catalyst is present in amount of at least 10 mol % based on glycerol carbonate. In another preferred embodiment, heating is accomplished by conventional thermal methods and the ionic liquid catalyst is present in amount of at least 10 mol % based on glycerol carbonate. In a further preferred embodiment, the process is conducted at a temperature of from 115° C. to 125° C. and the ionic liquid catalyst is present in amount of at least 10 mol % based on glycerol carbonate. In yet another preferred embodiment, heating is accomplished by microwave heating in a microwave reactor, the ionic liquid catalyst is present in amount of at least 10 mol % based on glycerol carbonate and the reaction is conducted in the presence of less than 5 wt % solvent, for example 2 wt %, 1 wt % or 0 wt %. In a particularly preferred embodiment, heating is accomplished by microwave heating in a microwave reactor, the process is conducted at a temperature of from 115° C. to 125° C., the amount of ionic liquid catalyst is at least 10 mol % based on glycerol carbonate and the reaction is conducted in the presence of less than 5 wt % solvent, for example 2 wt %, 1 wt % or 0 wt %.

The present invention will now be illustrated by way of the following examples and with reference to the following figures:

FIG. 1: Graphical representation of effect of temperature on conversion and selectivity as reported in Gade et al;

FIG. 2: Graphical representation of effect of GL:DMC ratio on conversion and selectivity as reported in in Gade et al; and

FIG. 3: Graphical representation of the effect of temperature (microwave heating with 15 minutes hold time) on glycidol selectivity for a glycidol synthesis according to the present invention wherein GL:DMC ratio is 1:5 using tributylmethylammonium methylcarbonate, 1-butyl-1-methylpyrrolidium methylcarbonate or tetramethylammonium hydroxide as ionic liquid catalyst.

### EXAMPLES

### Preparation of Ionic Liquids

Tetramethylammonium hydroxide was prepared from a commercially available 25% solution of aqueous tetramethylammonium solution. Water was removed from the solution using a rotary evaporator.

Tributylmethylammonium methylcarbonate and 1-butyl-1-methylpyrrolidinium methylcarbonate were prepared according to the microwave-assisted synthesis of methylcarbonate salts reported in Holbrey et al., Green Chem., 2010, 12, 407-413.

Tributylamine (1.854 g, 10 mmol), DMC (0.90 g, 10 mmol) and methanol (2 ml) were added to 10 ml glass microwave process vial together with a magnetic stirring bar before the vial was sealed and placed inside a CEM Explorer microwave reactor. The solution was heated at 160° C. for 1 hour hold time with magnetic stirring. Tributylmethylammonium methylcarbonate was isolated after removal of the volatile solvent and excess DMC under reduced pressure.

1-butylpyrrolidine (1.272 g, 10 mmol), DMC (0.90 g, 10 mmol) and methanol (2 ml) were added to 10 ml glass microwave process vial together with a magnetic stirring bar before the vial was sealed and placed inside a CEM Explorer microwave reactor. The solution was heated at 140° C. for 1 hour hold time with magnetic stirring. 1-butyl-1-methylpyrrolidinium methylcarbonate was isolated after removal of the volatile solvent and excess DMC under reduced pressure.

Microwave Reactions

Either Anton Paar: Monowave 300 or CEM Explorer microwave reactors were used for performing the microwave reactions, operating at a frequency of 2450 MHz with a maximum power output of 80 W. The ingredients were added to a 10 ml glass microwave process vial together with a magnetic stirrer bar before the vial was sealed and placed inside the reactor. Samples were then run for a predetermined time at a specified hold temperature. Run times referred to below, unless otherwise indicated, refer to the time a sample is held at a particular temperature, and not the total irradiation time.

Analysis of Product Samples

Following the reaction, samples were analysed by gas chromatography (GC) using an Agilent 6890N gas chromatograph with a HP-Innowax capillary column employing a He carrier gas operated according to the following: i) flow rate of 0.7 cm³ min⁻¹ at 50° C. for one minute; ii) linear gradient of 25° C. min⁻¹ to 200° C.; iii) linear gradient of 3° C. min⁻¹ from 200° C. to 230° C.; and iv) 18 minutes hold at 230° C.

### Example 1

1-butyl-1-methylpyrrolidinium methylcarbonate (0.02173 g, 0.1 mmol) was combined with glycerol (0.093 g, 1 mmol) and dimethylcarbonate (0.45 g, 5 mmol) in a 20 ml sealed glass tube with a pressure rating of 1000 kPa (10 bar), along with a magnetic stirrer bar. The sealed glass tube was placed in an oil bath pre-heated to 120° C. and stirred for 15 minutes with vigorous magnetic stirring. The glass tube was then removed from the oil bath and allowed to cool to room temperature before a sample extracted for gas chromatography (GC) analysis.

### Example 2

The process of Example 1 was repeated, except that the reaction was heated for 30 minutes at 120° C. Catalyst loading was kept constant at 10 mol % based on glycerol and the same molar ratio of glycerol:dimethyl carbonate was employed (1:5).

### Example 3

1-butyl-1-methylpyrrolidinium methylcarbonate (0.02173 g, 0.1 mmol) was combined with glycerol (0.093 g, 1 mmol) and dimethylcarbonate (0.45 g, 5 mmol) in a 10 ml glass microwave process vial, along with a magnetic stirrer bar, before the vial was sealed. The sample was placed inside a CEM Explorer microwave reactor heated with magnetic stirring for a hold time of 15 minutes at 120° C. and a pressure of 550 kPa (5.5 bar), before the reaction mixture was analysed directly by gas chromatography (GC).

### Example 4

The process of Example 3 was repeated, except that tributylmethylammonium methyl carbonate was used in place of 1-butyl-1-methylpyrrolidinium methylcarbonate. Catalyst loading was kept constant at 10 mol % based on glycerol and the same molar ratio of glycerol:dimethyl carbonate was employed (1:5).

### Example 5

The process of Example 3 was repeated, except that tetramethylammonium hydroxide was used in place of 1-butyl-1-methylpyrrolidinium methylcarbonate. Catalyst loading was kept constant at 10 mol % based on glycerol and the same molar ratio of glycerol:dimethyl carbonate was employed (1:5).

### Example 6

The process of Example 3 was repeated, except that a molar ratio of glycerol:dimethyl carbonate of 1:8 was used. Catalyst loading was kept constant at 10 mol % based on glycerol.

### Example 7

The processes of Examples 3 to 5 were repeated for a range of different hold temperatures (100° C., 140° C. and 160° C.). Catalyst loading was kept constant at 10 mol % based on glycerol and the same molar ratio of glycerol:dimethyl carbonate was employed (1:5) in each case.

### Comparative Example 1

The process of Example 3 was repeated, except that a molar ratio of glycerol:dimethyl carbonate of 1:15 was used. Catalyst loading was kept constant at 10 mol % based on glycerol.

### Comparative Example 2

Glycerol (0.093 g, 1 mmol) and dimethylcarbonate (0.45 g, 5 mmol) were both added to a 10 ml glass microwave process vial, along with a magnetic stirrer bar, before the vial was sealed. No ionic liquid catalyst was included in this reaction. The sample was placed inside an Anton Paar: Monowave 300 microwave reactor and run for 15 minutes at 160° C., before the reaction mixture was analysed directly by gas chromatography (GC).

Table 1 below shows the results of Examples 1 to 7, Comparative Examples 1 and 2 and the results of Run 1, 2 and 3 of Table 2 of Gade et al. The data in Table 2 of Gade et al were compiled from experiments involving a one-pot, synthesis of glycidol from glycerol (21.7 mmol) and dimethylcarbonate (21.7 mmol to 65.19 mmol) in the presence of tetramethylammonium hydroxide ionic liquid catalyst (0.217 mmol). These prior art reactions were performed at a temperature of 80° C. for a period of 90 minutes using thermal heating.

The results in Table 1 (corresponding to Entries 6, 7 and 10 to 19) have also been used for generating a graphical representation (FIG. 3).

TABLE 1

| | | | | | GD | GC |
|---|---|---|---|---|---|---|
| | | Temp. | GL:DMC | Conversion | selectivity | selectivity |
| Entry | Catalyst | (° C.) | ratio | of GL (%) | (%) | (%) |
| 1¹ | tetramethylammonium | 80° C. | 1:1 | 45 | 51 | 39 |
| | hydroxide | | | | | |
| 2¹ | tetramethylammonium | 80° C. | 1:2 | 74 | 55 | 40 |
| | hydroxide | | | | | |
| 3¹ | tetramethylammonium | 80° C. | 1:3 | 97 | 52 | 46 |
| | hydroxide | | | | | |
| 4² | 1-butyl-1- | 120° C. | 1:5 | 100 | 85 | 15 |
| | methylpyrrolidinium | | | | | |
| | methylcarbonate | | | | | |
| 5³ | 1-butyl-1- | 120° C. | 1:5 | 100 | 86 | 12 |
| | methylpyrrolidinium | | | | | |
| | methylcarbonate | | | | | |
| 6⁴ | 1-butyl-1- | 100° C. | 1:5 | 100 | 79 | 21 |
| | methylpyrrolidinium | | | | | |
| | methylcarbonate | | | | | |
| 7⁴ | 1-butyl-1- | 120° C. | 1:5 | 100 | 90 | 8 |
| | methylpyrrolidinium | | | | | |
| | methylcarbonate | | | | | |
| 8⁴ | 1-butyl-1- | 120° C. | 1:8 | 100 | 89 | — |
| | methylpyrrolidinium | | | | | |
| | methylcarbonate | | | | | |
| 9^{1, 4} | 1-butyl-1- | 120° C. | 1:15 | 100 | 41 | 30 |
| | methylpyrrolidinium | | | | | |
| | methylcarbonate | | | | | |
| 10⁴ | 1-butyl-1- | 140° C. | 1:5 | 100 | 79 | 1 |
| | methylpyrrolidinium | | | | | |
| | methylcarbonate | | | | | |
| 11⁴ | 1-butyl-1- | 160° C. | 1:5 | 100 | 76 | 0 |
| | methylpyrrolidinium | | | | | |
| | methylcarbonate | | | | | |
| 12⁴ | tetramethylammonium | 100° C. | 1:5 | 100 | 79 | 0 |
| | hydroxide | | | | | |
| 13⁴ | tetramethylammonium | 120° C. | 1:5 | 97 | 82 | 15 |
| | hydroxide | | | | | |
| 14⁴ | tetramethylammonium | 140° C. | 1:5 | 100 | 65 | 21 |
| | hydroxide | | | | | |
| 15⁴ | tetramethylammonium | 160° C. | 1:5 | 100 | 58 | 0 |
| | hydroxide | | | | | |
| 16⁴ | tributylmethylammonium | 100° C. | 1:5 | 78 | 45 | 32 |
| | methylcarbonate | | | | | |
| 17⁴ | tributylmethylammonium | 120° C. | 1:5 | 96 | 83 | 13 |
| | methylcarbonate | | | | | |
| 18⁴ | tributylmethylammonium | 140° C. | 1:5 | 100 | 69 | 21 |
| | methylcarbonate | | | | | |
| 19⁴ | tributylmethylammonium | 160° C. | 1:5 | 100 | 39 | 0 |
| | methylcarbonate | | | | | |
| 20¹ | None | 160° C. | 1:5 | 92 | 0 | 89 |

¹Not of the invention
²Reaction time = 15 minutes; Heating = oil bath
³Reaction time = 30 minutes; Heating = oil bath
⁴Microwave heating

The results of Table 1 show a surprisingly high rate of conversion and selectivity for glycidol achieved in a process according to the present invention (Entries 4 to 8 and 10 to 19), obtainable within a short reaction time. For instance, a GL conversion of 100% and a GD selectivity of 85% is obtained when GL and DMC, in a GL:DMC molar ratio of 1:5, are reacted in the presence of 1-butyl-1-methylpyrrolidinium methylcarbonate catalyst for 15 minutes at 120° C. using heat from an oil bath (Entry 4).

A GL conversion of 100% and a GD selectivity of 90% is obtained when GL and DMC, in a GL:DMC molar ratio of 1:5, are reacted in the presence of a 1-butyl-1-methylpyrrolidinium methylcarbonate catalyst at 120° C. in a microwave for a hold time of 15 minutes (Entry 7). A GL conversion of 97% and a GD selectivity of 82% is obtained when GL and DMC, in a GL:DMC molar ratio of 1:5, are reacted in the presence of a tetramethylammonium hydroxide catalyst at 120° C. in a microwave for a hold time of 15 minutes (Entry 13).

The results for Entry 5 in Table 1 demonstrate that although high glycerol conversion is obtainable in a microwave reaction, despite the absence of ionic liquid, there is no selectivity for glycidol and the formation of glycerol carbonate predominates.

Further embodiments relating to the present invention are also described below by means of the following clauses:

Clause 1. A one-pot synthetic process for the preparation of glycidol comprising the reaction of glycerol and dimethyl carbonate in the presence of an ionic liquid catalyst having the formula:

[Cat⁺][X⁻]
wherein: [Cat⁺] represents one or more cationic species, and
   [X⁻] represents one or more anionic species;
      wherein the reaction is conducted in a microwave reactor at a temperature of from 100° C. to 160° C.; and preferably wherein the molar ratio of glycerol to dimethylcarbonate is from 1:4 to 1:10.

Clause 2. A one-pot synthetic process for the preparation of glycidol comprising the reaction of glycerol and dimethyl carbonate in the presence of an ionic liquid catalyst having the formula:

[Cat⁺][X⁻]
wherein: [Cat⁺] represents one or more cationic species, and
   [X⁻] represents one or more anionic species;
      wherein the reaction is conducted in a microwave reactor and wherein the molar ratio of glycerol to dimethylcarbonate is from 1:4 to 1:10; and preferably wherein the reaction is conducted in a microwave reactor at a temperature of from 100° C. to 160° C.

Clause 3. The process according to Clause 1 or Clause 2, wherein the reaction is conducted at a temperature of from 110° C. to 140° C.

Clause 4. The process according to any of Clauses 1 to 3, wherein the reaction is conducted at a temperature of from 115° C. to 130° C.

Clause 5. The process according to any of Clauses 1 to 4, wherein the reaction is conducted at a temperature of from 115° C. to 125° C.

Clause 6. The process according to any of Clauses 1 to 5, wherein the molar ratio of glycerol to dimethylcarbonate is from 1:5 to 1:8.

Clause 7. The process according to any of Clauses 1 to 6, wherein the molar ratio of glycerol to dimethylcarbonate is 1:5.

Clause 8. The process according to any of Clauses 1 to 7, wherein the amount of ionic liquid catalyst is at least 2 mol % based on glycerol.

Clause 9. The process according to any of Clauses 1 to 8, wherein the amount of ionic liquid catalyst is at least 5 mol % based on glycerol.

Clause 10. The process according to any of Clauses 1 to 9, wherein the amount of ionic liquid catalyst is at least 8 mol % based on glycerol.

Clause 11. The process according to any of Clauses 1 to 10, wherein the amount of ionic liquid catalyst is at least 10 mol % based on glycerol.

Clause 12. The process according to any of Clauses 1 to 11, wherein [Cat⁺] comprises a cationic species selected from: ammonium, benzimidazolium, benzofuranium, benzothiophenium, benzotriazolium, borolium, cinnolinium, diazabicyclodecenium, diazabicyclononenium, 1,4-diazabicyclo[2.2.2]octanium, diazabicyclo-undecenium, dithiazolium, furanium, guanidinium, imidazolium, indazolium, indolinium, indolium, morpholinium, oxaborolium, oxaphospholium, oxazinium, oxazolium, iso-oxazolium, oxothiazolium, phospholium, phosphonium, phthalazinium, piperazinium, piperidinium, pyranium, pyrazinium, pyrazolium, pyridazinium, pyridinium, pyrimidinium, pyrrolidinium, pyrrolium, quinazolinium, quinolinium, iso-quinolinium, quinoxalinium, quinuclidinium, selenazolium, sulfonium, tetrazolium, thiadiazolium, iso-thiadiazolium, thiazinium, thiazolium, iso-thiazolium, thiophenium, thiuronium, triazinium, triazolium, iso-triazolium, and uronium.

Clause 13. The process according to any of Clauses 1 to 12, wherein [Cat⁺] comprises an acyclic cation selected from:

[N(R^{a})(R^{b})(R^{c})(R^{d})]⁺,[P(R^{a})(R^{b})(R^{c})(R^{d})]⁺, and [S(R^{a})(R^{b})(R^{c})]⁺,
wherein: R^{a}, R^{b}, R^{c}, and R^{d} are each independently selected from a C₁ to C₃₀, straight chain or branched alkyl group, a C₃ to C₈ cycloalkyl group, or a C₆ to C₁₀ aryl group; and wherein said alkyl, cycloalkyl or aryl groups are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₋₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

Clause 14. The process according to Clause 13, wherein [Cat⁺] comprises a a cation selected from:

[N(R^{a})(R^{b})(R^{c})(R^{d})]⁺,
wherein: R^{a}, R^{b}, R^{c}, and R^{d} are as defined in Clause 13.

Clause 15. The process according to Clause 14, wherein [Cat⁺] comprises a a cation selected from:

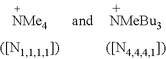

Clause 16. The process according to Clause 12, wherein [Cat⁺] comprises an aromatic heterocyclic cationic species selected from: benzimidazolium, benzofuranium, benzothiophenium, benzotriazolium, cinnolinium, diazabicyclodecenium, diazabicyclononenium, diazabicyclo-undecenium, dithiazolium, imidazolium, indazolium, indolinium, indolium, oxazinium, oxazolium, iso-oxazolium, oxathiazolium, phthalazinium, pyrazinium, pyrazolium, pyridazinium, pyridinium, pyrimidinium, quinazolinium, quinolinium, iso-quinolinium, quinoxalinium, tetrazolium, thiadiazolium, iso-thiadiazolium, thiazinium, thiazolium, iso-thiazolium, triazinium, triazolium, and iso-triazolium.

Clause 17. The process according to Clause 12, wherein [Cat⁺] comprises a saturated heterocyclic cation selected from cyclic ammonium, 1,4-diazabicyclo[2.2.2]octanium, morpholinium, cyclic phosphonium, piperazinium, piperidinium, quinuclidinium, and cyclic sulfonium.

Clause 18. The process according to Clause 16, wherein [Cat⁺] comprises a saturated heterocyclic cation having the formula:

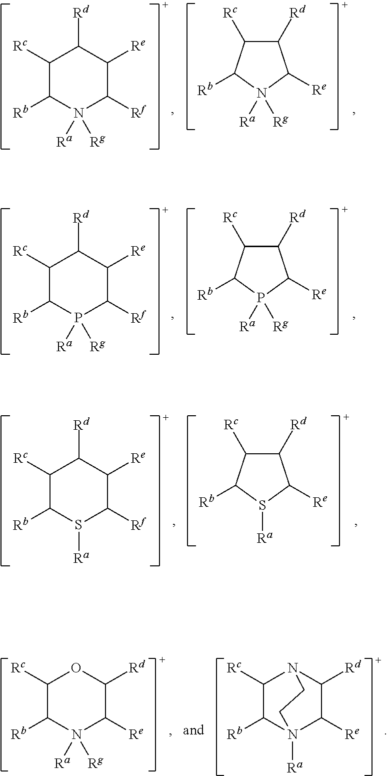

wherein: R^{a}, R^{b}, R^{c}, R^{d}, R^{e}, R^{f} and R^{g} are each independently selected from hydrogen, a C₁ to C₃₀, straight chain or branched alkyl group, a C₃ to C₈ cycloalkyl group, or a C₆ to C₁₀ aryl group, or any two of R^{b}, R^{c}, R^{d}, R^{e} and R^{f} attached to adjacent carbon atoms form a methylene chain —(CH₂)_{q}— wherein q is from 3 to 6; and wherein said alkyl, cycloalkyl or aryl groups or said methylene chain are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

Clause 19. The process according to Clause 18, wherein [Cat⁺] comprises a saturated heterocyclic cation having the formula:

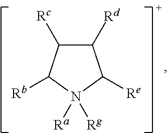

wherein: R^{a}, R^{b}, R^{c}, R^{d}, R^{e}, R^{f}, and R^{g} are as defined in Clause 18.

Clause 20. The process according to Clause 19, wherein [Cat⁺] comprises a saturated heterocyclic cation having the formula:

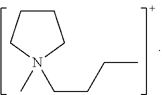

Clause 21. The process according to any of Clauses 1 to 20, wherein [X⁻] comprises one or more anions selected from hydroxides, halides, perhalides, pseudohalides, sulphates, sulphites, sulfonates, sulfonimides, phosphates, phosphites, phosphonates, methides, borates, carboxylates, azolates, carbonates, carbamates, thiophosphates, thiocarboxylates, thiocarbamates, thiocarbonates, xanthates, thiosulfonates, thiosulfates, nitrate, nitrite, perchlorate, halometallates, amino acids and borates.

Clause 22. The process according to Clause 21, wherein [X⁻] comprises a carbonate anion selected from [R²CO₃]⁻; wherein R² is selected from methyl, ethyl, n-propyl, n-butyl.

Clause 23. The process according to Clause 21, wherein [X⁻] comprises an anion selected from [CO₃]²⁻, [HCO₃]⁻, [MeCO₃]⁻, [OH]⁻, and [SH]⁻.

Clause 24. The process according to Clause 23, wherein [X⁻] comprises an anion selected from [MeCO₃]⁻ and [OH]⁻.

Clause 25. The process according to any of Clauses 1 to 11, wherein the ionic liquid is tributylmethylammonium methylcarbonate.

Clause 26. The process according to any of Clauses 1 to 11, wherein the ionic liquid is 1-butyl-1-methylpyrrolidinium methylcarbonate.

Clause 27. The process according to any of Clauses 1 to 11, wherein the ionic liquid is tetramethylammonium hydroxide.

1. A one-pot synthetic process for the preparation of glycidol comprising the reaction of glycerol and dimethyl carbonate in the presence of an ionic liquid catalyst having the formula:
[Cat⁺][X⁻]
wherein: [Cat⁺] represents one or more cationic species, and
[X⁻] represents one or more anionic species;
wherein the reaction is conducted at a temperature of from 100° C. to 160° C. and wherein the molar ratio of glycerol to dimethylcarbonate is from 1:4 to 1:10.

2. The process according to claim 1, wherein the molar ratio of glycerol to dimethylcarbonate is from 1:5 to 1:8.

3. The process according to claim 1 or claim 2, wherein the reaction is conducted at a temperature of from 110° C. to 140° C.

4. The process according to any of claims 1 to 3, wherein the reaction is conducted at a temperature of from 115° C. to 130° C.

5. The process according to any of claims 1 to 4, wherein the reaction is conducted at a temperature of from 115° C. to 125° C.

6. The process according to any of claims 1 to 5, wherein the amount of ionic liquid catalyst is at least 2 mol % based on glycerol.

7. The process according to any of claims 1 to 6, wherein the amount of ionic liquid catalyst is at least 5 mol % based on glycerol.

8. The process according to any of claims 1 to 7, wherein the amount of ionic liquid catalyst is at least 8 mol % based on glycerol.

9. The process according to any of claims 1 to 8, wherein the amount of ionic liquid catalyst is at least 10 mol % based on glycerol.

10. The process according to any of claims 1 to 9, wherein [Cat⁺] comprises a cationic species selected from: ammonium, benzimidazolium, benzofuranium, benzothiophenium, benzotriazolium, borolium, cinnolinium, diazabicyclodecenium, diazabicyclononenium, 1,4-diazabicyclo[2.2.2]octanium, diazabicyclo-undecenium, dithiazolium, furanium, guanidinium, imidazolium, indazolium, indolinium, indolium, morpholinium, oxaborolium, oxaphospholium, oxazinium, oxazolium, iso-oxazolium, oxothiazolium, phospholium, phosphonium, phthalazinium, piperazinium, piperidinium, pyranium, pyrazinium, pyrazolium, pyridazinium, pyridinium, pyrimidinium, pyrrolidinium, pyrrolium, quinazolinium, quinolinium, iso-quinolinium, quinoxalinium, quinuclidinium, selenazolium, sulfonium, tetrazolium, thiadiazolium, iso-thiadiazolium, thiazinium, thiazolium, iso-thiazolium, thiophenium, thiuronium, triazinium, triazolium, iso-triazolium, and uronium.

11. The process according to any of claims 1 to 10, wherein [Cat⁺] comprises an acyclic cation selected from:
[N(R^{a})(R^{b})(R^{c})(R^{d})]⁺,[P(R^{a})(R^{b})(R^{c})(R^{d})]⁺, and [S(R^{a})(R^{b})(R^{c})]⁺,
wherein: R^{a}, R^{b}, R^{c}, and R^{d} are each independently selected from a C₁ to C₃₀, straight chain or branched alkyl group, a C₃ to C₈ cycloalkyl group, or a C₆ to C₁₀ aryl group; and wherein said alkyl, cycloalkyl or aryl groups are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₋₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

12. The process according to claim 11, wherein [Cat⁺] comprises a a cation selected from:
[N(R^{a})(R^{b})(R^{c})(R^{d})]⁺,
wherein: R^{a}, R^{b}, R^{c}, and R^{d} are as defined in claim 11.

13. The process according to claim 12, wherein [Cat⁺] comprises a a cation selected from:

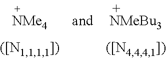

14. The process according to claim 10, wherein [Cat⁺] comprises an aromatic heterocyclic cationic species selected from: benzimidazolium, benzofuranium, benzothiophenium, benzotriazolium, cinnolinium, diazabicyclodecenium, diazabicyclononenium, diazabicyclo-undecenium, dithiazolium, imidazolium, indazolium, indolinium, indolium, oxazinium, oxazolium, iso-oxazolium, oxathiazolium, phthalazinium, pyrazinium, pyrazolium, pyridazinium, pyridinium, pyrimidinium, quinazolinium, quinolinium, iso-quinolinium, quinoxalinium, tetrazolium, thiadiazolium, iso-thiadiazolium, thiazinium, thiazolium, iso-thiazolium, triazinium, triazolium, and iso-triazolium.

15. The process according to claim 10, wherein [Cat⁺] comprises a saturated heterocyclic cation selected from cyclic ammonium, 1,4-diazabicyclo[2.2.2]octanium, morpholinium, cyclic phosphonium, piperazinium, piperidinium, quinuclidinium, and cyclic sulfonium.

16. The process according to claim 15, wherein [Cat⁺] comprises a saturated heterocyclic cation having the formula: 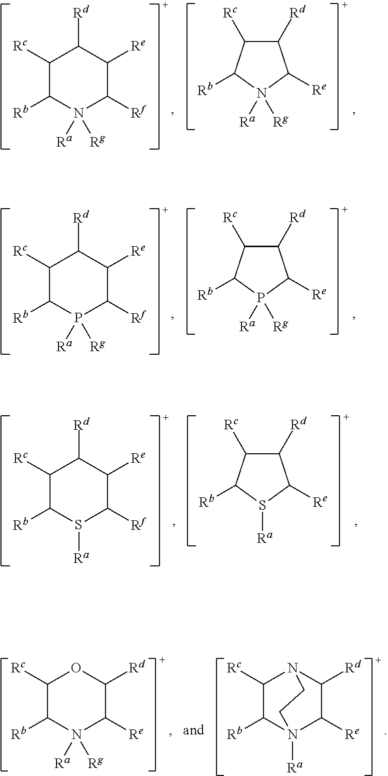
wherein: R^{a}, R^{b}, R^{c}, R^{d}, R^{e}, R^{f} and R^{g} are each independently selected from hydrogen, a C₁ to C₃₀, straight chain or branched alkyl group, a C₃ to C₈ cycloalkyl group, or a C₆ to C₁₀ aryl group, or any two of R^{b}, R^{c}, R^{d}, R^{e} and R^{f} attached to adjacent carbon atoms form a methylene chain —(CH₂)_{q}— wherein q is from 3 to 6; and wherein said alkyl, cycloalkyl or aryl groups or said methylene chain are unsubstituted or may be substituted by one to three groups selected from: C₁ to C₆ alkoxy, C₂ to C₁₂ alkoxyalkoxy, C₃ to C₈ cycloalkyl, C₆ to C₁₀ aryl, C₇ to C₁₀ alkaryl, C₇ to C₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁ to C₆)alkyl, —S(O)O(C₁ to C₆)alkyl, —OS(O)(C₁ to C₆)alkyl, —S(C₁ to C₆)alkyl, —S—S(C₁ to C₆alkyl), —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁ to C₆ alkyl.

17. The process according to claim 16, wherein [Cat⁺] comprises a saturated heterocyclic cation having the formula: 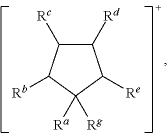
wherein: R^{a}, R^{b}, R^{c}, R^{d}, R^{e}, R^{f}, and R^{g} are as defined in claim 16.

18. The process according to claim 17, wherein [Cat⁺] comprises a saturated heterocyclic cation having the formula:

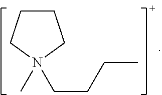

19. The process according to any of claims 1 to 18, wherein [X⁻] comprises one or more anions selected from hydroxides, halides, perhalides, pseudohalides, sulphates, sulphites, sulfonates, sulfonimides, phosphates, phosphites, phosphonates, methides, borates, carboxylates, azolates, carbonates, carbamates, thiophosphates, thiocarboxylates, thiocarbamates, thiocarbonates, xanthates, thiosulfonates, thiosulfates, nitrate, nitrite, perchlorate, halometallates, amino acids and borates.

20. The process according to claim 19, wherein [X⁻] comprises a carbonate anion selected from [R²CO₃]⁻; wherein R² is selected from methyl, ethyl, n-propyl, n-butyl.

21. The process according to claim 19, wherein [X⁻] comprises an anion selected from [CO₃]²⁻, [HCO₃]⁻, [MeCO₃]⁻, [OH]⁻, and [SH]⁻.

22. The process according to claim 21, wherein [X⁻] comprises an anion selected from [MeCO₃]⁻ and [OH]⁻.

23. The process according to any of claims 1 to 9, wherein the ionic liquid is tributylmethylammonium methylcarbonate.

24. The process according to any of claims 1 to 9, wherein the ionic liquid is 1-butyl-1-methylpyrrolidinium methylcarbonate.

25. The process according to any of claims 1 to 9, wherein the ionic liquid is tetramethylammonium hydroxide.

26. The process according to any of claims 1 to 25, wherein the reaction is heated by conventional thermal methods.

27. The process according to any of claims 1 to 25, wherein the reaction is heated by means of a microwave reactor.

28. A one-pot synthetic process for the preparation of glycidol comprising the reaction of glycerol and dimethyl carbonate in the presence of an ionic liquid catalyst having the formula:
[Cat⁺][X⁻]
wherein: [Cat⁺] represents one or more cationic species, and
[X⁻] represents one or more anionic species;
wherein the reaction is conducted at a temperature of from 100° C. to 160° C.; and wherein the ionic liquid catalyst is present in an amount of at least 8 mol % based on glycerol.

29. The process according to claim 28 wherein the ionic liquid catalyst is as defined in any of claims 10 to 25.

30. The process according to claim 28 or claim 29 wherein the reaction is conducted at a temperature as defined in any of claims 3 to 5.

31. The process according to any of claims 28 to 30, wherein the molar ratio of glycerol to dimethylcarbonate is as defined in claim 1 or claim 2.

32. The process according to any of claims 28 to 31, wherein the ionic liquid catalyst is present in an amount of at least 10 mol % based on glycerol.

33. The process according to any of claims 28 to 32, wherein the reaction is heated by conventional thermal methods.

34. The process according to any of claims 28 to 32, wherein the reaction is heated by means of a microwave reactor.

35. A one-pot synthetic process for the preparation of glycidol comprising the reaction of glycerol and dimethyl carbonate in the presence of an ionic liquid catalyst having the formula:
[Cat⁺][X⁻]
wherein: [Cat⁺] represents one or more cationic species, and
[X⁻] represents one or more anionic species;
wherein the reaction is conducted in a microwave at a temperature of from 100° C. to 160° C.; and preferably wherein the molar ratio of glycerol to dimethylcarbonate is from 1:4 to 1:10.

36. A one-pot synthetic process for the preparation of glycidol comprising the reaction of glycerol and dimethyl carbonate in the presence of an ionic liquid catalyst having the formula:
[Cat⁺][X⁻]
wherein: [Cat⁺] represents one or more cationic species, and
[X⁻] represents one or more anionic species;
wherein the reaction is conducted in a microwave and wherein the molar ratio of glycerol to dimethylcarbonate is from 1:4 to 1:10; and preferably wherein the temperature is from 100° C. to 160° C.

37. The process according to claim 35 or claim 36 wherein the ionic liquid catalyst is as defined in any of claims 10 to 25.

38. The process according to any of claims 35 to 37 wherein the reaction is conducted at a temperature as defined in any of claims 3 to 5.

39. The process according to any of claims 35 to 38 wherein the molar ratio of glycerol to dimethylcarbonate is from 1:5 to 1:8.

40. The process according to any of claims 35 to 39, wherein the ionic liquid catalyst is present in an amount as defined in any of claims 6 to 9.

41. A process substantially as defined in any of claims 1 to 40 and with reference to the Examples and/or Figures.
